(19) United States
(12) Patent Application Publication
(10) Pub. No.: US 2022/0225916 A1
(43) Pub. Date: Jul. 21, 2022

(54) APPARATUS AND METHOD FOR NON-CONTACT MEASURING MOMENTUM BY USING IR-UWB RADAR

(71) Applicant: IUCF-HYU (INDUSTRY-UNIVERSITY COOPERATION FOUNDATION HANYANG UNIVERSITY), Seoul (KR)

(72) Inventors: Sung Ho CHO, Seoul (KR); Hyun Kyung PARK, Seoul (KR); Won Hyuk LEE, Seoul (KR); Dae Hyeon YIM, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 17/614,775

(22) PCT Filed: May 15, 2020
(86) PCT No.: PCT/KR2020/006422

§ 371 Date: Nov. 29, 2021

(30) Foreign Application Priority Data
May 28, 2019 (KR) 10-2019-0062366

(51) Int. Cl. A61B 5/16; G01S 13/02; A61B 5/0507; G01S 13/52; G01S 13/06

(52) U.S. Cl. CPC A61B 5/168; G01S 13/0209; G01S 13/88; G01S 13/52; G01S 13/06; A61B 5/0507

(57) ABSTRACT

The present invention can provide an apparatus and a method for non-contact measuring momentum, the apparatus and the method being capable of quantitatively measuring the dynamic activity and static activity of a subject by using a plurality of IR-UWB radars and, particularly, capable of measuring, in real time, some activities of the human body and general activity of the human body by distinguishing moving activity of the subject from non-moving activity. Therefore, the activity of a subject is non-contact measured so as to minimize user inconvenience, thereby facilitating performance of an ADHD test even for a subject in a young age group, and thus ADHD can be diagnosed early.

### TECHNICAL FIELD

The present invention relates to an apparatus and a method for measuring an activity amount, and more particularly, to an apparatus and a method for measuring an activity amount in a non-contact manner using an impulse radio ultra-wideband (IR-UWB) radar.

### BACKGROUND ART

Movement disorders are clinical syndromes, such as Parkinson's disease, dystonia, tic disorder, Tourette's disorder, and attention-deficit/hyperactivity disorder (ADHD), which cause excessive movement or lack of voluntary/involuntary movement.

In the existing evaluation of movement disorder, in general, a comprehensive attention test (CAT) has been performed on a subject, and a medical specialist has observed a patient's concentration state during a test, thereby determining the movement disorder. Although determination criteria are established in a manual, since determination depends on subjective rating scales and clinical observations, results are often vague due to movement of a subject.

Accordingly, there has been a need for a method of quantitatively measuring movement of a subject during a test, and thus, techniques using an infrared camera, a three-dimensional (3D) camera, an actigraphy, and the like have been proposed.

In the case of the technique using the infrared camera, since movement of a specific part of a subject is reflected, an activity cannot be measured, and it is not easy to distinguish movement disorder from other neurodevelopmental disorders. In the case of the 3D camera, there is a problem in that a range is limited due to an angle of view and a measurable distance is in a short range of several meters.

Meanwhile, the actigraphy is a type of acceleration sensor developed to measure the quality of sleep, and the actigraphy can track an activity amount of a subject as well as a position thereof and thus is currently most commonly used to measure excessive movement in ADHD. However, since the actigraphy is a contact type sensor, the actigraphy not only causes uncomfortableness to a user but also has a limitation in that actigraphy cannot reflect movement of an entire body when worn on a specific part of a body such as an ankle or a wrist.

### DISCLOSURE

### Technical Problem

The present invention is directed to providing an apparatus and a method for measuring an activity amount, which are capable of accurately measuring an activity of a subject in a non-contact manner.

The present invention is also directed to providing an apparatus and a method for measuring an activity amount, which are capable of measuring sedentary movement of a subject as well as spatial movement thereof.

### Technical Solution

According to one embodiment of the present invention, an apparatus for measuring an activity amount includes a signal acquisition unit configured to acquire reception signals by sampling signals received when impulse signals are emitted from a plurality of impulse radio ultra-wideband (IR-UWB) radars disposed at predetermined positions and are reflected and configured to remove clutter included in the reception signal to acquire a background subtraction signal, a spatial movement amount measurement unit configured to calculate a subject distance from each of the plurality of IR-UWB radars to a subject from the background subtraction signal to determine a position of the subject and calculate an acceleration according to position movement of the subject to obtain a spatial movement measurement value, a sedentary movement amount measurement unit configured to obtain an activity change amount for a magnitude difference between the background subtraction signal and a previous background subtraction signal, accumulate the activity change amount to obtain accumulated change amounts for each of the plurality of IR-UWB radars, and obtain a predetermined statistical value among the obtained accumulated change amounts for each of the plurality of IR-UWB radars as a sedentary movement measurement value, and an activity amount output unit configured to output the spatial movement measurement value and the sedentary movement measurement value in a predetermined manner.

The signal acquisition unit may include a radar unit which includes the plurality of IR-UWB radars and acquires the plurality of reception signals by sampling the signals received when the impulse signals are emitted from the plurality of IR-UWB radars and are reflected, a background subtraction unit which removes the clutter from the reception signal to acquire the background subtraction signal, and a threshold value setting unit which acquires accumulated background subtraction signals by accumulating the background subtraction signal acquired during a predetermined period in a state in which the subject is not positioned, and sets a threshold value using the accumulated background subtraction signals according to a constant false alarm rate (CFAR) algorithm.

The spatial movement amount measurement unit may include a signal detection unit configured to detect a background subtraction signal that is greater than the threshold value and extract a minimum distance index among distance indexes set according to a sampling order in the detected background subtraction signal, a distance determination unit configured to calculate the subject distance from each of the plurality of IR-UWB radars to the subject from the minimum distance index, a position estimation unit configured to estimate the position of the subject from the subject distance from each of the plurality of IR-UWB radars to the subject according to a least-squares method, an acceleration calculation unit configured to calculate a movement speed and an acceleration of the subject from the position of the subject estimated over time, and a spatial movement determination unit configured to calculate the spatial movement measurement value by applying a predetermined spatial movement parameter to the acceleration.

The sedentary movement measurement unit may include a change amount accumulation unit configured to detect the background subtraction signal that is greater than the threshold value and calculate and accumulate the activity change amount for the magnitude difference between the detected background subtraction signal and the previous background subtraction signal to obtain the accumulated change amounts for each of the plurality of IR-UWB radars, and a sedentary movement determination unit configured to obtain a median value among the accumulated change amounts for each of the plurality of IR-UWB radars to extract the median value as the sedentary movement measurement value.

The activity amount output unit may receive the spatial movement measurement value and the sedentary movement measurement value, when the spatial movement measurement value is greater than or equal to a predetermined reference spatial movement value, the activity amount output unit may output the spatial movement measurement value as an activity value of the subject, and when the spatial movement measurement value is less than the predetermined reference spatial movement value, the activity amount output unit may output the sedentary movement value as the activity value of the subject.

According to another embodiment of the present invention, a method of measuring an activity amount includes sampling signals, which are received when impulse signals are emitted from a plurality of IR-UWB radars disposed at predetermined positions and are reflected, to acquire reception signals and removing clutter included in the reception signal to acquire a background subtraction signal, calculating a subject distance from each of the plurality of IR-UWB radars to a subject from the background subtraction signal to determine a position of the subject and calculating an acceleration according to position movement of the subject to obtain a spatial movement measurement value, obtaining an activity change amount for a magnitude difference between the background subtraction signal and a previous background subtraction signal, accumulating the activity change amount to obtain accumulated change amounts for each of the plurality of IR-UWB radars, and obtaining a predetermined statistical value among the obtained accumulated change amounts for each of the plurality of IR-UWB radars as a sedentary movement measurement value, and outputting the spatial movement measurement value and the sedentary movement measurement value in a predetermined manner.

### Advantageous Effects

Therefore, in an apparatus and a method for measuring an activity amount according to embodiments of the present invention, it is possible to accurately and quantitatively measure both spatial movement and sedentary movement of a subject using a plurality of impulse radio ultra-wideband (IR-UWB) radars. In addition, since user's inconvenience can be minimized by measuring an activity of a subject in a non-contact manner, an attention-deficit/hyperactivity disorder (ADHD) test can be easily performed on even subjects with a young age, which makes it possible to early diagnose ADHD. In addition, the apparatus and the method for measuring an activity amount can be used for measuring an activity amount of old people living alone or for preventing the solitary death.

### DESCRIPTION OF DRAWINGS

FIG. 1 illustrates a schematic structure of an apparatus for measuring an activity amount using an impulse radio ultra-wideband (IR-UWB) radar according to one embodiment of the present invention.

FIGS. 2 and 3 illustrate examples of a measurement environment of the apparatus for measuring an activity amount using an IR-UWB radar according to the present embodiment.

FIG. 4 shows an example of a sedentary movement amount measurement result for each scenario of an apparatus for measuring an activity amount according to the present embodiment.

FIG. 5 shows an example of a spatial movement amount measurement result for each scenario of an apparatus for measuring an activity amount according to the present embodiment.

FIGS. 6 and 7 show results of comparing sedentary and spatial movement amount measurement results of an apparatus for measuring an activity amount according to the present embodiment with results measured with an actigraphy.

FIG. 8 illustrates a method of measuring an activity amount using an IR-UWB radar according to one embodiment of the present invention.

### MODES OF THE INVENTION

In order to fully understand the present invention, operational advantages of the present invention, and objects achieved by the implementation of the present invention, the accompanying drawings illustrating exemplary embodiments of the present invention and the contents described in the accompanying drawings need to be referred to.

Hereinafter, the present invention will be described in detail by describing exemplary embodiments of the present invention with reference to the accompanying drawing. However, the present invention may be implemented in various forms and is not limited to the embodiments described herein. In order to clearly describe the present invention, portions that are not associated with a description will be omitted, and like reference numerals denote like members.

Throughout the present specification, unless explicitly described to the contrary, “comprising” any components will be understood to imply the inclusion of other elements rather than the exclusion of any other elements. In addition, a term “˜unit”, “˜er/or,” “module,” “block,” or the like described in the specification means a processing unit of at least one function or operation and may be implemented by hardware or software or a combination of hardware and software.

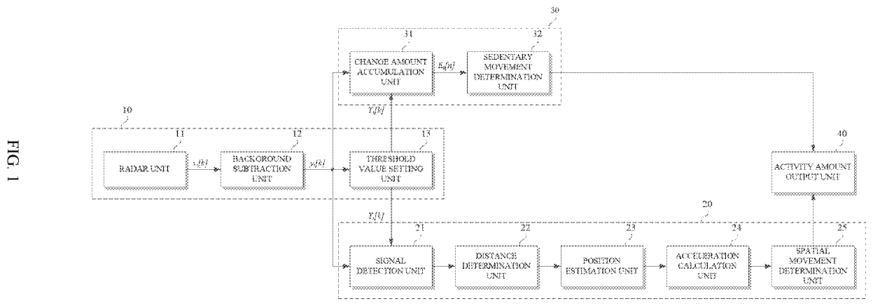

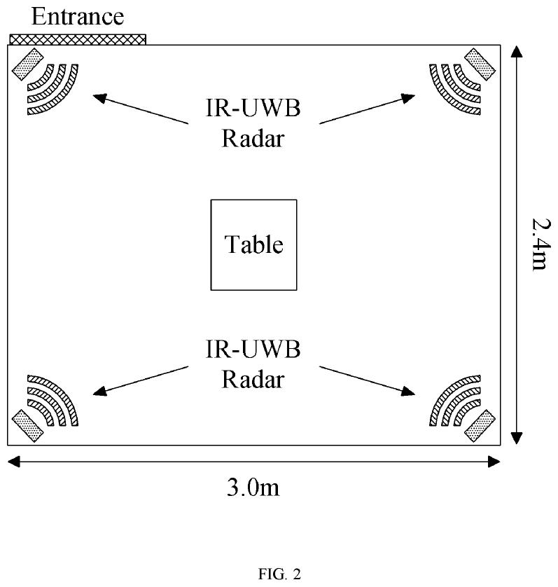

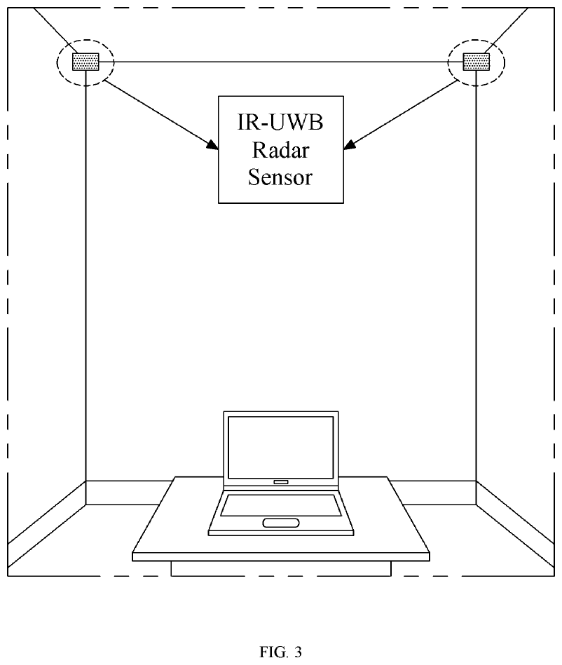

FIG. 1 illustrates a schematic structure of an apparatus for measuring an activity amount using an impulse radio ultra-wideband (IR-UWB) radar according to one embodiment of the present invention, and FIGS. 2 and 3 illustrate examples of a measurement environment of the apparatus for measuring an activity amount using an IR-UWB radar according to the present embodiment.

Referring to FIG. 1, the apparatus for measuring an activity amount using an IR-UWB radar according to the present embodiment includes a signal acquisition unit 10, a spatial movement measurement unit 20, a sedentary movement measurement unit 30, and an activity amount output unit 40.

The signal acquisition unit 10 acquires a signal for measuring an activity amount of a subject in a non-contact manner. In the present embodiment, the signal acquisition unit 10 includes a plurality of IR-UWB radars, acquires a sampling signal xᵢ[k] from a reception signal received by the plurality of IR-UWB radars, acquires a background subtraction signal yᵢ[k] by removing clutter from the sampling signal xᵢ[k], and sets a threshold value T_{i,n}[k] for determining whether the subject is active from the acquired background subtraction signal yᵢ[k].

The signal acquisition unit 10 may include a radar unit 11, a background subtraction unit 12, and a threshold value setting unit 13. The radar unit 11 includes the plurality of IR-UWB radars disposed at predetermined positions. Each of the plurality of IR-UWB radars emits a predetermined impulse signal s[k], acquires the reception signal xᵢ[k] including noise and the impulse signal, which is emitted and reflected by a surrounding environment, and transmits the acquired reception signal xᵢ[k] to the background subtraction unit 12.

Since the IR-UWB radar uses a UWB frequency that is harmless to the human body, the IR-UWB radar can detect a subject in a non-contact manner without interference from other sensors. In addition, even when signals are emitted and received with very low power, the IR-UWB can provide sufficient range and resolution in an indoor environment. In particular, since the IR-UWB radar can provide precision enough to be used to measure respiration or heart rate in a medical field, the IR-UWB radar can measure even a minute activity of a subject and can be installed so as to be unrecognizable by the subject due to excellent transmittance thereof.

As an example, as shown in FIGS. 2 and 3, the plurality of IR-UWB radars may be disposed at ceiling positions of four corners in a quadrangular indoor environment in which a table, at which a subject is to be placed, is disposed at a central portion thereof.

In general, a comprehensive attention test (CAT) is performed indoors such that a subject is influenced by an external environment as little as possible and is easy to observe. Accordingly, in the present embodiment, as an example, the plurality of IR-UWB radars are disposed so as to measure an activity of a subject in the quadrangular indoor environment and are disposed at the ceiling positions of the four corners so as to measure the activity of the subject as accurately as possible as well as so as to not draw a subject's attention as much as possible. However, the number and arrangement positions of the IR-UWB radars included in the radar unit 11 may be variously adjusted.

In FIGS. 2 and 3, although the IR-UWB radar is disposed in a form that is recognizable by the subject for convenience, the IR-UWB radar may be disposed to be unrecognizable by the subject as described above.

The impulse signal s[k] emitted from each of the plurality of IR-UWB radars is delayed and attenuated by being reflected through various paths by a wall, a subject, and various objects in an indoor environment and is mixed with noise N[k] introduced thereinto, and the mixed signal is received by each of the plurality of IR-UWB radars. Therefore, the reception signal xᵢ[k] received and sampled by an i^{th} radar among the plurality of IR-UWB radars may be represented as in Equation 1.

$${x}_{i} [ k ] = {\sum }_{m = 1}^{{N}_{path}} {a}_{m , i} s [ k - {τ}_{m , i} ] + 𝒩 [ k ] \qquad [ Equation 1 ]$$

Here, k may be a sampling index according to a period for which the reception signal xᵢ[k] is sampled, may also be referred to as a distance index, and may be expressed as a natural number from zero to a maximum observable distance index L_{signal} in a designated environment. Nₚₐₜₕ denotes the number of paths through which the emitted impulse signal s[k] is reflected and received, and a_{m,i} and τ_{m,i} respectively denote a scale value and a delay value when the impulse signal s[k] is received by the i^{th} radar along an m^{th} path.

The background subtraction unit 12 removes clutter from the reception signal xᵢ[k] to acquire the background subtraction signal yᵢ[k]. As described above, in an indoor environment, the impulse signal s[k] is reflected by various objects including a wall, that is, a background, other than a subject and is received as the reception signal xᵢ[k], and a component of the reception signal xᵢ[k], which is reflected by the background and received, is called a clutter signal. Since the apparatus for measuring an activity according to the present embodiment should measure an activity amount of a subject, the clutter signal excluding a component reflected by the subject should be removed from the reception signal xᵢ[k].

In general, since an object corresponding to a background is fixed, the background subtraction signal yᵢ[k] may be acquired by removing the clutter signal from the reception signal xᵢ[k] as in Equation 2.

$${y}_{i , n} [ k ] = {x}_{i , n} [ k ] - {C}_{i . n} [ k ] , \\ {C}_{i , n} [ k ] = α {C}_{i , n - 1} [ k ] + ( 1 - α ) {x}_{i , n} [ k ] \qquad [ Equation 2 ]$$

Here, n denotes a sequence index of a reception signal acquired by each radar, C_{i,n}[k] denotes a clutter signal included in a reception signal x_{i,n}[k] in an n^{th} sequence acquired by the i^{th} IR-UWB radar, and α is a real number with a predetermined value between 0 and 1.

The background subtraction signal yᵢ[k], from which the clutter signal C_{i,n}[k], which is a component caused by a background, is removed from the reception signal xᵢ[k], may be the sum of a signal component \hat{r}ᵢ[k] caused by a subject and noise Nᵢ[k] and represented as in Equation 3.

$${y}_{i} [ k ] = {\hat{r}}_{i} [ k ] + {𝒩}_{i} [ k ] \qquad [ Equation 3 ]$$

The background subtraction unit 12 provides the acquired background subtraction signal yᵢ[k] to each of the spatial movement measurement unit 20 and the sedentary movement measurement unit 30.

The threshold value setting unit 13 sets a threshold value T_{i,n}[k] for determining whether an activity of the subject is spatial movement or sedentary movement. As described above, the background subtraction signal yᵢ[k] includes the signal component \hat{r}ᵢ[k] caused by the subject and the noise Nᵢ[k]. The signal component \hat{r}ᵢ[k] caused by the subject may include a spatial movement component due to movement of the subject and a sedentary movement component due to movement of a specific part excluding the spatial movement component of the subject.

The threshold value setting unit 13 sets the threshold value T_{i,n}[k] so as to not erroneously determine that the subject has performed an activity due to the noise Nᵢ[k] even though the subject does not perform the activity.

The threshold value setting unit 13 receives and accumulates the background subtraction signal yᵢ[k] during a predetermined period in a state in which the subject is not positioned and acquires the accumulated background subtraction signals yᵢ[k] (=[y_{i,0}[k], y_{i,1}[k], . . . , y_{i,2}[k], . . . , and y_{i,Nc}[k]^{T}), wherein Nc denotes the number of the accumulated background subtraction signals yᵢ[k]. This is to enable the threshold value setting unit 13 to set the threshold value Tᵢ[k] adaptively suitable for an activity amount measurement environment that is variously implementable.

The threshold value setting unit 13 may set the threshold value Tᵢ[k] using the acquired accumulated background subtraction signals yᵢ[k] as in Equation 4 according to a constant false alarm rate (CFAR) algorithm.

$${T}_{i} [ k ] = β {σ}_{i} [ k ] + {μ}_{i} [ k ] \qquad [ Equation 4 ]$$

Here, i is a radar identifier, β is a parameter for adjusting the threshold value Tᵢ[k], and μᵢ[k] and σᵢ[k] denote a mean and a standard deviation of the accumulated background subtraction signals yᵢ[k], respectively.

The threshold value setting unit 13 provides the set threshold value Tᵢ[k] to each of the spatial movement measurement unit 20 and the sedentary movement measurement unit 30.

The spatial movement measurement unit 20 receives the background subtraction signal y_{i,n}[k] from the signal acquisition unit 10 and measures a spatial movement amount representing a movement amount of the subject. In order to accurately detect a spatial movement amount of the subject, the spatial movement measurement unit 20 calculates distances from the plurality of IR-UWB radars to the subject from the background subtraction signal y_{i,n}[k] using a CFAR algorithm and detects a change in position of the subject determined according to the calculated distances from the plurality of IR-UWB to the subject, thereby measuring spatial movement of the subject.

The spatial movement measurement unit 20 may include a signal detection unit 21, a distance determination unit 22, a position estimation unit 23, an acceleration calculation unit 24, and a spatial movement determination unit 25.

The signal detection unit 21 receives the background subtraction signal yᵢ[k], detects a signal yᵢ[k] that is greater than the threshold value Tᵢ[k] set by the threshold value setting unit 13 (yᵢ[k]>Tᵢ[k]), and extracts a minimum distance index among distance indexes k of the detected background subtraction signal yᵢ[k]. That is, the signal detection unit 21 extracts a minimum distance index k_{i,min} extractable for each of the plurality of IR-UWB radars. In this case, as for the background subtraction signal yᵢ[k] corresponding to at least one IR-UWB radar among the plurality of IR-UWB radars, a signal that is greater than the threshold value Tᵢ[k] may not be detected, and in this case, the background subtraction signal yi[k] acquired from the at least one IR-UWB radar is ignored.

When the signal detection unit 21 extracts the minimum distance index k_{i,min}, the distance determination unit 22 calculates a subject distance dᵢ from each of the plurality of IR-UWB radars to the subject using the extracted minimum distance index k_{i,min}.

The distance determination unit 22 may calculate and obtain the subject distance dᵢ as dᵢ=c/fₛ×k_{i,min} according to a sampling frequency fₛ. Here, c is the speed of light.

The position estimation unit 23 estimates a position of the subject using the subject distance dᵢ calculated by the distance determination unit 22.

However, the position of the subject may not be specified only with the subject distance dᵢ obtained from one IR-UWB radar, and the position of the subject may be specified only when the subject distances dᵢ are obtained from at least two IR-UWB radars.

Accordingly, the position estimation unit 23 estimates the position of the subject when the subject distances dᵢ from at least two IR-UWB radars to the subject are applied.

A position (xᵢ, yᵢ, zᵢ) of the subject relative to the i^{th} IR-UWB radar in a three-dimensional space may be represented using the subject distance dᵢ as in Equation 5.

$${( x - {x}_{i} )}^{2} + {( y - {y}_{i} )}^{2} + {( z - {z}_{i} )}^{2} = {d}_{i}^{2} \qquad [ Equation 5 ]$$

When subject distances dᵢ and dₘ from an l′^{h} IR-UWB radar and an m^{th} IR-UWB radar are calculated by the distance determination unit 22 and applied, Equation 6 may be derived from Equation 5.

$$2 x ( {x}_{m} - {x}_{l} ) + 2 y ( {y}_{m} - {y}_{l} ) + 2 z ( {z}_{m} - {z}_{l} ) = {d}_{l}^{2} - {d}_{m}^{2} - {x}_{l}^{2} + {x}_{m}^{2} - {y}_{l}^{2} + {y}_{m}^{2} - {z}_{l}^{2} + {z}_{m}^{2} \qquad [ Equation 6 ]$$

Equation 6 may be solved using a least-squares method, and to this end, Equation 6 may be converted into the form of a matrix equation such as Ax=b. Here, A and b of the matrix equation may be represented by Equation 7.

$$A = 2 [ \begin{matrix}{x}_{1} - {x}_{0} & {y}_{1} - {y}_{0} & {z}_{1} - {z}_{0} \\ {x}_{2} - {x}_{1} & {y}_{1} - {y}_{0} & {z}_{1} - {z}_{0} \\ … & … & … \\ {x}_{{N}_{r}} - {x}_{{N}_{r} - 1} & {y}_{{N}_{r}} - {y}_{{N}_{r} - 1} & {z}_{{N}_{r}} - {z}_{{N}_{r} - 1}\end{matrix} ] , \\ b = [ \begin{matrix}{C}_{1} \\ {C}_{2} \\ … \\ {C}_{{N}_{r}}\end{matrix} ] \qquad [ Equation 8 ]$$

In Equation 7, the right side of Equation 6 is replaced with d²ᵢ−d²ᵢ₋₁−x²ᵢ+ₓ₂ᵢ₋₁−y²ᵢ+y²ᵢ₋₁−z²ᵢ+z²ᵢ₋₁ due to Cᵢ. Since a solution of the least-squares method is known as the right side of Equation 8, a position p of the subject may be obtained as p=[xₜ, yₜ, zₜ]^{T}.

$$p = {( {A}^{T} A )}^{- 1} {A}^{T} b \qquad [ Equation 8 ]$$

A position p[n] of the subject over time may be expressed as p[n]=[xₜ[n], yₜ[n], zₜ[n]]^{T}.

The acceleration calculation unit 24 calculates a velocity v[n] and an acceleration a[n] for movement of the subject from the position p[n] of the subject over time obtained by the position estimation unit 23 according to Equation 9.

$$v [ n ] = ( p [ n ] - p [ n - 1 ] ) / {t}_{r} \\ a [ n ] = ( v [ n ] - v [ n - 1 ] ) / {t}_{r} \qquad [ Equation 9 ]$$

Here, tᵣ may denote a sampling period for position data of the subject as a radar observation period, and initial values of the velocity v[n] and the acceleration a[n] may be specified as v[0]=v[1]=a[0]=0.

The spatial movement amount determination unit 26 calculates a spatial movement measurement value Mₛₚₐₜᵢₐₗ for spatial movement according to Equation 10 using the acceleration a[n] calculated in Equation 9.

$${M}_{spatial} [ n ] = β \cdot a [ n ] = γ ( p [ n ] - 2 p [ n - 1 ] + p [ n - 2 ] ) \qquad [ Equation 10 ]$$

Here, β and γ are predetermined values as spatial movement parameters.

Meanwhile, the subject may perform various activities in a fixed position without moving or perform activities separate from movement while moving, and when such a sedentary movement amount can be measured together with a spatial movement amount, more accurate results can be derived in a CAT.

Accordingly, the sedentary movement measurement unit 30 receives the background subtraction signal yᵢ[k] from the signal acquisition unit 10 and measures a sedentary movement amount of the subject. In the present embodiment, a sedentary movement amount may include all activities such as various activities in a stationary position and local activities of a body during movement except for dynamic movements indicating position movement of the subject.

The sedentary movement measurement unit 30 may include a change amount accumulation unit 31 and a sedentary movement determination unit 32.

The change amount accumulation unit 31 receives the background subtraction signal y_{i,n}[k] according to the sequence index n of the reception signal x_{i,n}[k] acquired by the radar and obtains an activity change amount g_{i,n}[k] for a magnitude difference between the received background subtraction signal y_{i,n}[k] and a previous background subtraction signal y_{i,n-1}[k]. In this case, in order to not erroneously determine an activity of the subject due to the noise Nᵢ[k], the change amount accumulation unit 31 also detects only the background subtraction signal yᵢ[k] that is greater than the threshold value Tᵢ[k] set by the threshold value setting unit 13 (yᵢ[k]>>Tᵢ[k]) to obtain the activity change amount g_{i,n}[k] and accumulates the obtained activity change amount g_{i,n}[k] to obtain accumulated change amounts Eᵢ[n] for each radar according to Equation 11.

$${E}_{1} [ n ] = {\sum }_{k = 0}^{{L}_{signal}} {g}_{i , n} [ k ] , \\ {g}_{i , n} [ k ] = \{ \begin{matrix}{\vert {y}_{i , n} [ k ] - {y}_{i , n - 1} [ k ] \vert}^{2} & if {y}_{i , n} [ k ] > {T}_{i} [ k ] \\ 0 & if {y}_{i , n} [ k ] \leq {T}_{i} [ k ]\end{matrix} . \qquad [ Equation 11 ]$$

The sedentary movement determination unit 32 calculates a sedentary movement measurement value M_{sedentary} for sedentary movement using the accumulated change amount Eᵢ[n] for each radar obtained by the change amount accumulation unit 31 according to Equation 12.

$${M}_{sedentary} [ n ] = Median ( {E}_{0} [ n ] , {E}_{1} [ n ] , {E}_{2} [ n ] , … , {E}_{{N}_{r}} [ n ] ) \qquad [ Equation 11 ]$$

Nᵣ is the number of the IR-UWB radars, and Median(•) is a median function.

A reason for the sedentary movement determination unit 32 to obtain a median value of the accumulated change amounts Eᵢ[n] for each radar as in Equation 11 is because, when the subject is too close to or too far from the radar, since the accumulated change amount Eᵢ[n] for each radar is measured too large or too small, the sedentary movement measurement value M_{sedentary} of the subject cannot be accurately determined. That is, the sedentary movement determination unit 32 selects the median value of the accumulated change amounts Eᵢ[n] for each radar as the sedentary movement measurement value M thereby increasing the reliability of the sedentary movement measurement value M_{sedentary}.

The activity amount output unit 40 outputs the spatial movement measurement value Mₛₚₐₜᵢₐₗ obtained by the spatial movement measurement unit 20 and the sedentary movement measurement value M_{sedentary} obtained by the sedentary movement measurement unit 30 in a predetermined manner. In this case, the activity amount output unit 40 may output the spatial movement measurement value Mₛₚₐₜᵢₐₗ and the sedentary movement measurement value M_{sedentary} in the form of a numerical value without change and may output the spatial movement measurement value Mₛₚₐₜᵢₐₗ and the sedentary movement measurement value M_{sedentary} in the form of separate graphs such that an activity amount of the subject can be easily observed.

As described above, in the present embodiment, the spatial movement measurement value Mₛₚₐₜᵢₐₗ is obtained based on a movement acceleration of the subject, and the sedentary movement measurement value M_{sedentary} is obtained based on a change amount of a magnitude of the accumulated background subtraction signals y_{i,n}[k].

Therefore, spatial movement indicating movement of the subject indicates relatively great movement as compared with sedentary movement indicating a standstill or movement of a specific part. In addition, the sedentary movement measurement value M_{sedentary}, which is obtained based on the activity change amount g_{i,n}[k] according to the magnitude difference between the background subtraction signal y_{i,n}[k] and the previous background subtraction signal y_{i,n-1}[k], is varied not only by sedentary movement of the subject but also by spatial movement thereof. Accordingly, when the spatial movement measurement value Mₛₚₐₜᵢₐₗ is than greater or equal to a predetermined reference spatial movement value, the activity amount output unit 40 may output the spatial movement measurement value Mₛₚₐₜᵢₐₗ as an activity value of the subject, and when the spatial movement measurement value Mₛₚₐₜᵢₐₗ is less than the reference spatial movement value, the activity amount output unit 40 may output the sedentary movement measurement value M_{sedentary} as the activity value of the subject. Here, the reference spatial movement value is a value set in order to prevent erroneous determination in which the subject has performed spatial movement even though the subject does not perform the spatial movement due to a measurement error.

In addition, when the spatial movement measurement value Mₛₚₐₜᵢₐₗ is greater than or equal to the reference spatial movement value, the activity amount output unit 40 may be configured to receive the accumulated change amount Eᵢ[n] for each radar, pre-estimate and subtract an accumulated change amount corresponding to the spatial movement measurement value Mₛₚₐₜᵢₐₗ to output an accumulated sedentary change amount. Here, since an accumulated change amount due to spatial movement has already been subtracted from the accumulated change amount Eᵢ[n] for each radar, even though the subject performs both dynamic and sedentary movements, the accumulated sedentary change amount includes only an accumulated change amount for sedentary movement. The sedentary movement measurement value M_{sedentary} for the accumulated sedentary change amount may also be obtained by extracting a median value as in Equation 11.

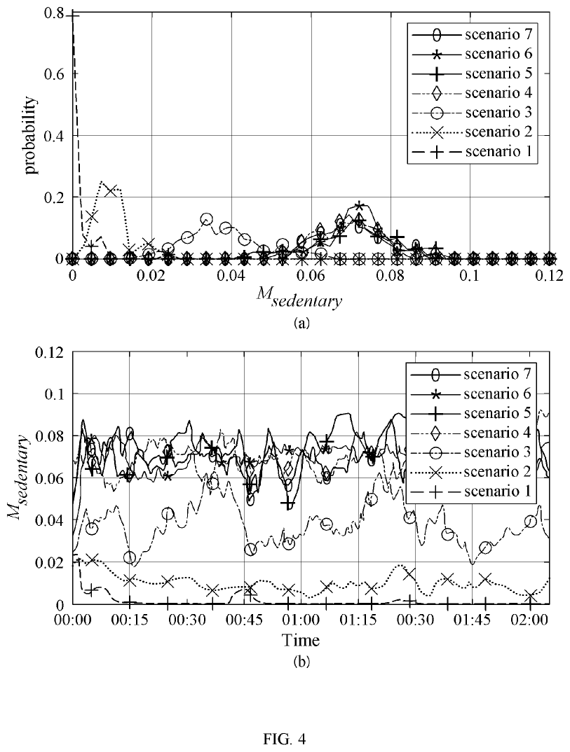

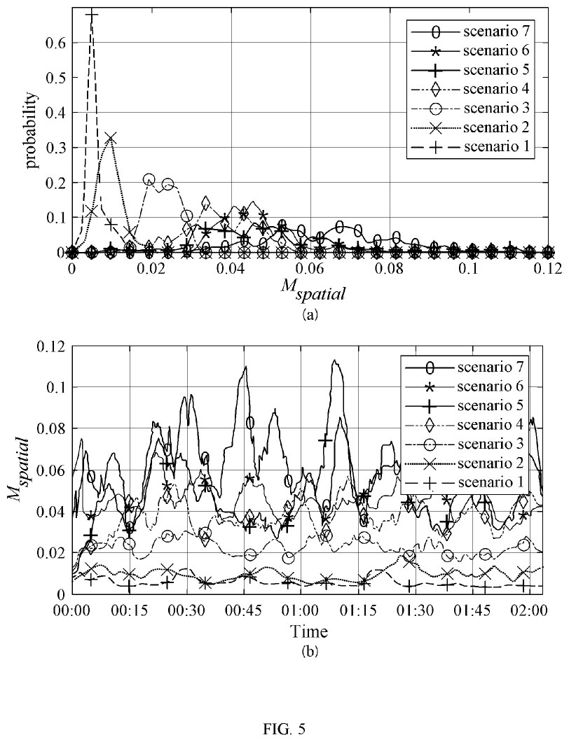

FIG. 4 shows an example of a sedentary movement amount measurement result for each scenario of an apparatus for measuring an activity amount according to the present embodiment, and FIG. 5 shows an example of a spatial movement amount measurement result for each scenario of an apparatus for measuring an activity amount according to the present embodiment.

FIGS. 4 and 5 show results in which the apparatus for measuring an activity amount according to the present embodiment measures spatial movement and sedentary movement of a subject in various situations. In order to verify the performance of the apparatus for measuring an activity amount, measurements were performed in situations according to the following seven scenarios.

1. When the subject sits down and concentrates on one thing.

2. When the subject performs relatively small movement in a sitting position.

3. When the subject performs relatively large movement in a sitting position.

4. When the subject walks slowly in a narrow-radius room.

5. When the subject walks slowly at a large radius in a room.

6. When the subject walks fast in a narrow-radius room.

7. When the subject walks fast at a large radius in a room.

Here, a center frequency of an IR-UWB radar was set to 8.748 GHz, and a bandwidth thereof was set to 1.5 GHz. Sampling was performed at a rate of 23.328 GS/s, and as shown in FIGS. 2 and 3, the IR-UWB radar was installed on a ceiling of each of four corners in an indoor space with a width of 2.4 m, a length of 3.0 m, and a height of 2.4 m. A table, at which the subject was placed, was disposed at a central portion of the indoor space.

FIGS. 4A and 5A show histograms of a sedentary movement measurement value M_{sedentary} and a spatial movement measurement value Mₛₚₐₜᵢₐₗ for each scenario, and FIGS. 4B and 5B show real time measurement values of the sedentary movement measurement value M_{sedentary} and the spatial movement measurement value Mₛₚₐₜᵢₐₗ for each scenario.

Referring to FIG. 4, in first to third scenarios, it can be seen that the sedentary movement measurement value M_{sedentary} is increased so that a result for each scenario is consistently and appropriately output similar to an expected result. On the other hand, in fourth to seventh scenarios, it can be seen that the real time measurement result appears similar to the spatial movement measurement value Mₛₚₐₜᵢₐₗ of FIG. 5 but is not output at a level sufficient to measure spatial movement.

Meanwhile, referring to FIG. 5, in the first to third scenarios, a difference between real time measurement results of the spatial movement measurement values Mₛₚₐₜᵢₐₗ is not great so that it is not easy to distinguish each scenario, but in the fourth to seventh scenarios, a difference between real time measurement results is great. Therefore, as shown in FIGS. 4 and 5, it can be seen that an activity degree of the subject can be accurately determined in real time from the sedentary movement measurement value M_{sedentary} and the spatial movement measurement value Mₛₚₐₜᵢₐₗ.

Tables 1 and 2 show a mean of sedentary movement measurement values M_{sedentary} and a mean of spatial movement measurement values Mₛₚₐₜᵢₐₗ of five subjects in the seven scenarios described above.

TABLE 1

Mean of M_{sedentary}

| Scenario | A | B | C | D | E | Total |
|---|---|---|---|---|---|---|
| 1 | 0.16 | 0.16 | 0.14 | 0.30 | 0.19 | 0.19 |
| 2 | 1.01 | 1.77 | 0.76 | 0.73 | 0.85 | 1.02 |
| 3 | 3.81 | 4.27 | 2.30 | 2.20 | 2.01 | 2.92 |
| 4 | 6.99 | 5.10 | 2.78 | 5.72 | 4.74 | 5.07 |
| 5 | 7.11 | 5.04 | 4.64 | 5.11 | 4.43 | 5.27 |
| 6 | 7.15 | 6.60 | 3.06 | 5.04 | 7.02 | 5.77 |
| 7 | 6.94 | 5.39 | 4.57 | 3.85 | 6.25 | 5.40 |

TABLE 2

Mean of Mₛₚₐₜᵢₐₗ

| Scenario | A | B | C | D | E | Total |
|---|---|---|---|---|---|---|
| 1 | 0.55 | 0.68 | 0.56 | 0.54 | 0.47 | 0.56 |
| 2 | 0.95 | 1.25 | 1.20 | 1.72 | 1.56 | 1.34 |
| 3 | 2.28 | 2.65 | 2.89 | 2.53 | 3.46 | 2.76 |
| 4 | 3.71 | 2.52 | 2.75 | 3.75 | 2.91 | 3.13 |
| 5 | 4.77 | 4.27 | 4.56 | 5.37 | 3.40 | 4.47 |
| 6 | 4.54 | 3.21 | 4.15 | 5.82 | 3.44 | 4.23 |
| 7 | 6.46 | 6.16 | 5.45 | 7.55 | 5.60 | 6.24 |

Referring to Tables 1 and 2, in the same scenario, it can be confirmed that similar sedentary movement measurement values M_{sedentary} and similar spatial movement measurement values Mₛₚₐₜᵢₐₗ are obtained in different subjects. This means that the sedentary movement measurement value M_{sedentary} and the spatial movement measurement value Mₛₚₐₜᵢₐₗ can be accurately obtained based only on an activity of the subject irrespective of the subject. However, in the case of subjects with a very different physical condition, such as children, a sedentary movement amount may be measured to be smaller than that of FIG. 4, but a spatial movement amount may be measured to be substantially the same as that of FIG. 4.

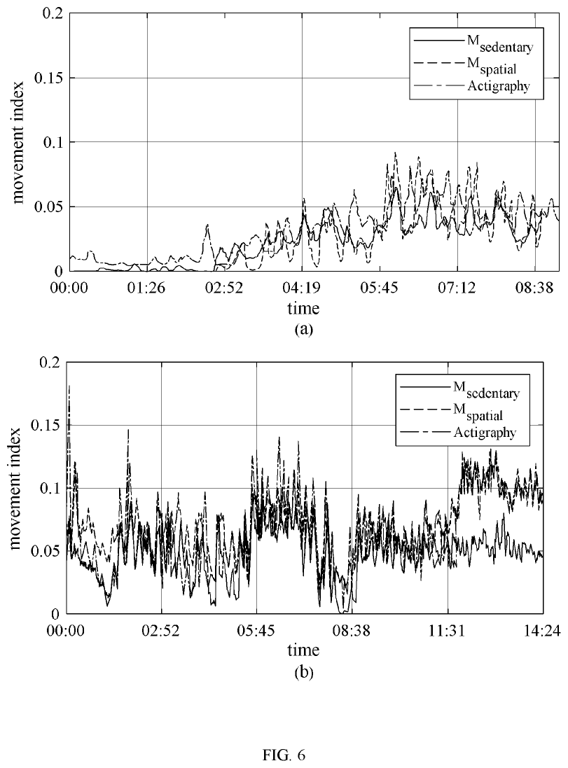

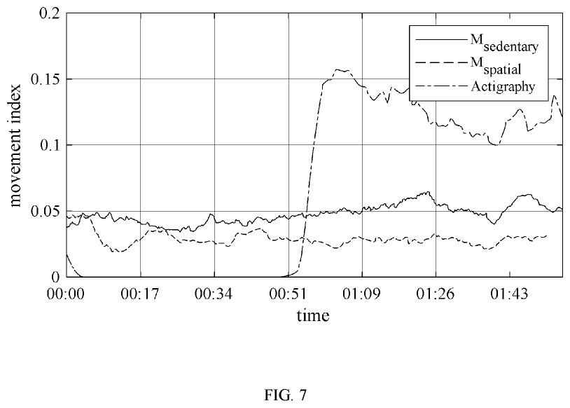

FIGS. 6 and 7 show results of comparing sedentary and spatial movement amount measurement results of an apparatus for measuring an activity amount according to the present embodiment with results measured with an actigraphy.

FIG. 6A shows a result of measuring an activity amount when a subject is active at a standstill, and FIG. 6B shows a result of measuring an activity amount when the subject is active while moving.

Referring to FIG. 6, the results measured with the actigraphy may appear similar to those of the apparatus for measuring an activity amount according to the present embodiment, but as shown in a section up to 2:50 of FIG. 6A, since the actigraphy measures an activity of the subject based on an acceleration sensor worn on a specific body part, there is a limitation in that an accurate activity amount cannot be measured when the specific body part is not active even though the subject is active.

FIG. 7 shows an extreme example for describing a measurement error of the actigraphy using an acceleration sensor worn on a specific body part. As described above, when other parts of the subject performs are active without an activity of a body part on which the acceleration sensor is worn or only the corresponding body part is active, there are problems in that the actigraphy may erroneously determine that there is no activity of the subject as shown in a section up to 00:51 in FIG. 7, and on the contrary, the actigraphy may erroneously determine that there is an excessive activity of the subject as shown in a section after 00:51 in FIG. 7. On the other hand, the apparatus for measuring an activity amount according to the present embodiment detects an activity amount of an entire body of the subject, thereby detecting an accurate activity amount.

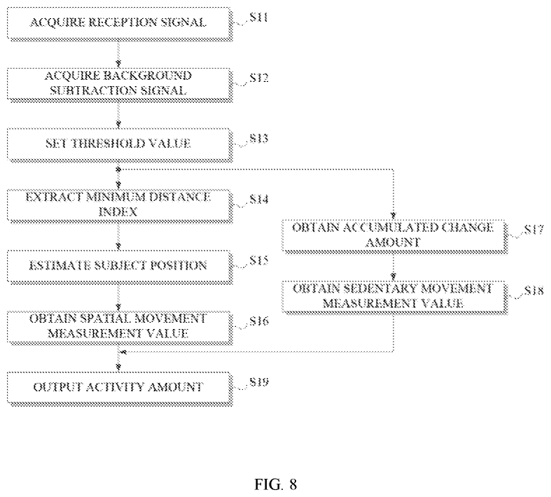

FIG. 8 illustrates a method of measuring an activity amount using an IR-UWB radar according to one embodiment of the present invention.

Describing the method of measuring an activity amount using an IR-UWB radar with reference to FIG. 1, first, a reception signal xᵢ[k] is acquired by sampling signals including noise and impulse signals s[k] that are emitted from a plurality of IR-UWB radars disposed at predetermined positions and are reflected from a surrounding environment (S11).

A clutter signal is removed from the reception signal xᵢ[k] to acquire a background subtraction signal yᵢ[k] (S12). When the background subtraction signal yᵢ[k] is acquired, a threshold value T_{i,n}[k] is set not to erroneously determine that a subject has performed an activity even though the subject does not perform the activity due to the noise Nᵢ[k]. Here, as an example, the threshold value T_{i,n}[k] may be set using the background subtraction signal yᵢ[k] accumulated during a predetermined period according to a CFAR algorithm.

When the background subtraction signal yᵢ[k] and the threshold value T_{i,n}[k] are obtained, in the method of measuring an activity, a spatial movement amount measurement operation and a sedentary movement amount measurement operation may be performed in parallel.

In the spatial movement measurement operation, first, a background subtraction signal that is greater than the threshold value Tᵢ[k] among the background subtraction signals yᵢ[k] for each of the plurality of IR-UWB radars is detected, and a minimum distance index k_{i,min} is extracted from among distance indexes k of the detected background subtraction signal yᵢ[k] (S14). A subject distance dᵢ from each of the plurality of IR-UWB radars to the subject is calculated using the extracted minimum distance index k_{i,min}, and a position of the subject is estimated from the calculated subject distance dᵢ from each of the plurality of IR-UWB radars to the subject (S15).

When the position of the subject is estimated, a velocity v[n] and an acceleration a[n] for movement of the subject are obtained from a position p[n] of the subject over time, and a spatial movement measurement value Mₛₚₐₜᵢₐₗ is obtained using the obtained acceleration according to Equation 10 (S16).

Meanwhile, in the sedentary movement amount measurement operation, an activity change amount g_{i,n}[k] for a magnitude difference between the applied background subtraction signal y_{i,n}[k] and a previous background subtraction signal y_{i,n-1}[k] is obtained, and the activity change amount g_{i,n}[k] is accumulated to obtain accumulated change amounts Eᵢ[n] for each radar according to Equation 11 (S17). In this case, after the background subtraction signal y_{i,n}[k] that is less than or equal to the threshold value Tᵢ[k] is removed, the activity change amount g_{i,n}[k] may be obtained.

When the accumulated change amount Eᵢ[n] for each of the plurality of IR-UWB radars is obtained, a median value of the acquired accumulated change amounts Eᵢ[n] is obtained as a sedentary movement measurement value M_{sedentary} (S18).

When the spatial movement measurement value Mₛₚₐₜᵢₐₗ and the sedentary movement measurement value M_{sedentary} are obtained, the obtained spatial movement measurement value Mₛₚₐₜᵢₐₗ and sedentary movement measurement value M_{sedentary} are output as activity amounts of the subject in a predetermined manner (S19).

Here, the spatial movement measurement value Mₛₚₐₜᵢₐₗ and the sedentary movement measurement value M_{sedentary} may be output as individual numerical values that each varies in real time and may also be output by being converted into the form of graph as shown in FIGS. 4 and 5.

In addition, in some cases, the accumulated change amount Eᵢ[n] for each radar may be received, and an accumulated change amount corresponding to the spatial movement measurement value Mₛₚₐₜᵢₐₗ may be pre-estimated and subtracted to output an accumulated sedentary change amount, and the obtained accumulated sedentary change amount may be output together with the spatial movement measurement value Mₛₚₐₜᵢₐₗ.

As a result, in an apparatus and a method for measuring an activity amount in a non-contact manner using an IR-UWB radar according to the present embodiment, it is possible to quantitatively measure spatial movement and sedentary movement of a subject using a plurality of IR-UWB radars. In particular, an overall activity, not just an activity of a part of a body, can be measured in real time by distinguishing moving and non-moving activities of a subject. In addition, since user's inconvenience can be minimized by measuring an activity of a subject in a non-contact manner, an attention-deficit/hyperactivity disorder (ADHD) test can be easily performed on even subjects with a low age, which makes it possible to early diagnose ADHD.

The method according to the present invention may be implemented by a computer program stored in a medium so as to execute the method in a computer. Here, a computer readable medium may be any available medium that can be accessed by a computer and may also include all computer storage media. The computer storage media include volatile and nonvolatile media and separable and inseparable media implemented in any method or technology for storage of information such as computer readable instructions, data structures, program modules, or other data and include a random access memory a read-only memory (ROM), a random access memory (RAM), a compact disk (CD)-ROM, a digital video disk (DVD)-ROM, magnetic tape, a floppy disk, and an optical data storage device.

Although the present invention has been described with reference to the embodiments shown in the drawings, this is merely exemplary, and it should be understood by those skilled in the art that various changes and modifications may be made thereto and other embodiments equivalent thereto are possible.

Accordingly, the true technical protection scope of the present invention should be determined by the technical spirit of the appended claims.

1. An apparatus for measuring an activity amount in a non-contact manner, the apparatus comprising:
a signal acquisition unit configured to acquire reception signals by sampling signals received when impulse signals are emitted from a plurality of impulse radio ultra-wideband (IR-UWB) radars disposed at predetermined positions and are reflected and configured to remove clutter included in the reception signal to acquire a background subtraction signal;
a spatial movement amount measurement unit configured to calculate a subject distance from each of the plurality of IR-UWB radars to a subject from the background subtraction signal to determine a position of the subject and calculate an acceleration according to position movement of the subject to obtain a spatial movement measurement value;
a sedentary movement amount measurement unit configured to obtain an activity change amount for a magnitude difference between the background subtraction signal and a previous background subtraction signal, accumulate the activity change amount to obtain accumulated change amounts for each of the plurality of IR-UWB radars, and obtain a predetermined statistical value among the obtained accumulated change amounts for each of the plurality of IR-UWB radars as a sedentary movement measurement value; and
an activity amount output unit configured to output the spatial movement measurement value and the sedentary movement measurement value in a predetermined manner.

2. The apparatus of claim 1, wherein the signal acquisition unit includes:
a radar unit which includes the plurality of IR-UWB radars and acquires the plurality of reception signals by sampling the signals received when the impulse signals are emitted from the plurality of IR-UWB radars and are reflected;
a background subtraction unit which removes the clutter from the reception signal to acquire the background subtraction signal; and
a threshold value setting unit which acquires accumulated background subtraction signals by accumulating the background subtraction signal acquired during a predetermined period in a state in which the subject is not positioned, and sets a threshold value using the accumulated background subtraction signals according to a constant false alarm rate (CFAR) algorithm.

3. The apparatus of claim 2, wherein the spatial movement amount measurement unit includes:
a signal detection unit configured to detect a background subtraction signal that is greater than the threshold value and extract a minimum distance index among distance indexes set according to a sampling order in the detected background subtraction signal;
a distance determination unit configured to calculate the subject distance from each of the plurality of IR-UWB radars to the subject from the minimum distance index;
a position estimation unit configured to estimate the position of the subject from the subject distance from each of the plurality of IR-UWB radars to the subject according to a least-squares method;
an acceleration calculation unit configured to calculate a movement speed and an acceleration of the subject from the position of the subject estimated over time; and
a spatial movement determination unit configured to calculate the spatial movement measurement value by applying a predetermined spatial movement parameter to the acceleration.

4. The apparatus of claim 3, wherein the sedentary movement measurement unit includes:
a change amount accumulation unit configured to detect the background subtraction signal that is greater than the threshold value and calculate and accumulate the activity change amount for the magnitude difference between the detected background subtraction signal and the previous background subtraction signal to obtain the accumulated change amounts for each of the plurality of IR-UWB radars; and
a sedentary movement determination unit configured to obtain a median value among the accumulated change amounts for each of the plurality of IR-UWB radars to extract the median value as the sedentary movement measurement value.

5. The apparatus of claim 4, wherein the activity amount output unit receives the spatial movement measurement value and the sedentary movement measurement value,
when the spatial movement measurement value is greater than or equal to a predetermined reference spatial movement value, the activity amount output unit outputs the spatial movement measurement value as an activity value of the subject, and
when the spatial movement measurement value is less than the predetermined reference spatial movement value, the activity amount output unit outputs the sedentary movement value as the activity value of the subject.

6. The apparatus of claim 4, wherein, when the spatial movement measurement value is greater than or equal to a predetermined reference spatial movement value, the activity amount output unit receives the accumulated change amounts for each of the plurality of IR-UWB radars, estimates and subtracts an accumulated change amount corresponding to the spatial movement measurement value to obtain an accumulated sedentary change amount, and outputs the spatial movement measurement value and the accumulated sedentary change amount together.

7. A method of measuring an activity amount in a non-contact manner, the method comprising:
sampling signals, which are received when impulse signals are emitted from a plurality of IR-UWB radars disposed at predetermined positions and are reflected, to acquire reception signals and removing clutter included in the reception signal to acquire a background subtraction signal;
calculating a subject distance from each of the plurality of IR-UWB radars to a subject from the background subtraction signal to determine a position of the subject and calculating an acceleration according to position movement of the subject to obtain a spatial movement measurement value;
obtaining an activity change amount for a magnitude difference between the background subtraction signal and a previous background subtraction signal, accumulating the activity change amount to obtain accumulated change amounts for each of the plurality of IR-UWB radars, and obtaining a predetermined statistical value among the obtained accumulated change amounts for each of the plurality of IR-UWB radars as a sedentary movement measurement value; and
outputting the spatial movement measurement value and the sedentary movement measurement value in a predetermined manner.

8. The method of claim 7, wherein the acquisition of the background subtraction signal includes:
sampling the signals, which are received when the impulse signals are emitted from the plurality of IR-UWB radars and are reflected, to acquire the plurality of reception signals;
removing the clutter from the reception signal to acquire the background subtraction signal; and
accumulating the background subtraction signal acquired during a predetermined period in a state in which the subject is not positioned and acquiring accumulated background subtraction signals and setting a threshold value using the accumulated background subtraction signals according to a constant false alarm rate (CFAR) algorithm.

9. The method of claim 8, wherein the obtainment of the spatial movement measurement value includes:
detecting a background subtraction signal that is greater than the threshold value and extracting a minimum distance index among distance indexes set according to a sampling order in the detected background subtraction signal;
calculating the subject distance from each of the plurality of IR-UWB radars to the subject from the minimum distance index;
estimating the position of the subject according to a least-squares method from the subject distance from each of the plurality of IR-UWB radars to the subject;
calculating a movement speed and an acceleration of the subject from the position of the subject estimated over time; and
calculating the spatial movement measurement value by applying a predetermined spatial movement parameter to the acceleration.

10. The method of claim 9, wherein the obtainment of the sedentary movement measurement value includes:
detecting the background subtraction signal that is greater than the threshold value and calculating and accumulating the activity change amount for the magnitude difference between the detected background subtraction signal and the previous background subtraction signal to obtain the accumulated change amounts for each of the plurality of IR-UWB radars; and
obtaining a median value among the accumulated change amounts for each of the plurality of IR-UWB radars to extract the median value as the sedentary movement measurement value.

11. The method of claim 10, wherein the outputting includes:
receiving the spatial movement measurement value and the sedentary movement measurement value, and when the spatial movement measurement value is greater than or equal to a predetermined reference spatial movement value, outputting the spatial movement measurement value as an activity value of the subject; and
when the spatial movement measurement value is less than the predetermined reference spatial movement value, outputting the sedentary movement value as the activity value of the subject.
